(19) United States
(12) Patent Application Publication
(10) Pub. No.: US 2016/0045444 A1
(43) Pub. Date: Feb. 18, 2016

(54) COMPOSITE FORMULATION COMPRISING A FILM COATING LAYER CONTAINING ROSUVASTATIN OR A PHARMACEUTICALLY ACCEPTABLE SALT THEREOF

(71) Applicant: HANMI PHARM. CO., LTD., Hwaseong-si, Gyeonggi-do (KR)

(72) Inventors: Yong Il KIM, Suwon-si (KR); Hayoung JEONG, Seoul (KR); Kyeong Soo KIM, Suwon-si (KR); Jae Hyun PARK, Suwon-si (KR); Jong Soo WOO, Suwon-si (KR)

(73) Assignee: HANMI PHARM. CO., LTD., Hwaseong-si, Gyeonggi-do (KR)

(21) Appl. No.: 14/778,678

(22) PCT Filed: Apr. 30, 2014
(86) PCT No.: PCT/KR2014/003859

§ 371 Date: Sep. 21, 2015

(30) Foreign Application Priority Data
May 6, 2013 (KR) 10-2013-0050949
Mar. 18, 2014 (KR) 10-2014-0031675

(51) Int. Cl. A61K 9/48; A61K 31/202; A61K 31/192; A61K 31/155; A61K 9/28; A61K 31/505

(52) U.S. Cl. CPC A61K 9/4866; A61K 9/4891; A61K 9/284; A61K 31/505; A61K 31/192; A61K 31/155; A61K 31/202; A61K 9/2813

(57) ABSTRACT

Provided is a composite formulation comprising: (i) a core including a first pharmacological component; and (ii) a film coating layer formed on a surface of the core, which contains rosuvastatin or a pharmaceutically acceptable salt thereof as a second pharmacological component and a polyvinyl alcohol-polyethylene glycol graft copolymer and polyvinyl alcohol as a coating material. In the composite formulation of the present invention, tension and fluidity of the film coating layer are excellent, and thus breakage and a defective ratio are low. Accordingly, a composite agent containing rosuvastatin effective to relieve and treat a hyperlipemia symptom, or its pharmaceutically acceptable salt can be provided with high efficiency, and is present in a composite formulation form, and thus compliance of a patient can be improved.

### FIELD OF THE INVENTION

The present invention relates to a composite formulation comprising a film coating layer containing rosuvastatin or a pharmaceutically acceptable salt thereof, a polyvinyl alcohol-polyethylene glycol graft copolymer, and polyvinyl alcohol.

### BACKGROUND OF THE INVENTION

Currently, one of the main interests in the field of medicine is to co-administer existing drugs having various mechanisms in order to maximize pharmacological activity and increase a therapeutic effect. For example, drugs having various mechanisms of action, such as statin- and fibrate-based drugs, are co-prescribed to patients with hyperlipidemia. Although the use of combination drugs exhibits high pharmacological efficiency, it has limitations in that usage is complicated and thus compliance of the patient is reduced.

Various research efforts have been carried out in order to overcome the limitations regarding compliance of the patient, and methods of simultaneous administration of drugs having various mechanisms of action and usages have been proposed so as to simplify the administration procedure for improved patient compliance.

Nevertheless, the method of simultaneous administration was unable to sufficiently resolve the compliance limitation, and there has been suggested a combination formulation comprising two or more of medications in a single formulation.

Early combination formulation models, e.g., a simple mix-type tablet or capsule, a multilayer type tablet and the like, have merits in that an idea thereof is easily obtained and production is performed with ease; but many limitations in stability due to interaction of two or more different active components were discovered. Because of such stability issues, early combination formulation models are inadequate to be developed into various types of composite formulation due to lack of versatility.

Korean Patent Application Laid-Open Publication No. 2006-0077812 discloses a composite formulation prepared by coating a general drug tablet with a film coating layer comprising another drug. The formulation has a merit in that interaction rarely occurs between drugs as compared to the aforementioned early formulations. In the same vein, Korean Patent Application Laid-Open Publication No. 2013-0039797 provides a composite formulation comprising a tablet encapsulated in a hard capsule, which has an improved stability by minimizing a contact area between two drugs.

These two inventions regarding the composite formulation exhibit excellent performance and possibility as the composite formulation. However, from the research result by the present inventors, it was confirmed that when the film coating layer including rosuvastatin calcium salt was applied on a capsule including a first pharmaceutically active ingredient, there was a fatal limitation.

In general, the film coating layer has tension over a certain level, and thus when deformation of a core and external physical stimulation occur, the film coating layer should essentially have a property of maintaining a shape. However, in the case of the film coating layer including rosuvastatin calcium salt, the film coating layer has insufficient tension due to physical properties of the corresponding component. The film coating layers formed by using a coating solution including rosuvastatin calcium salt and pharmaceutically extensively used single coating material such as Kollicoat® IR, polyvinyl alcohol, hypromellose, povidone and the like, on the hard capsule tend to be broken and stripped even by a weak impact, and has a high possibility of breakage during production, packaging, and distribution processes, and thus commercialization is judged to be difficult.

Accordingly, the present inventors have studied coating materials which can provide sufficient tension to the film coating layer including rosuvastatin or a pharmaceutically acceptable salt thereof and minimize breakage and defects, and a significantly improved result was obtained by using the coating materials with a polyvinyl alcohol-polyethylene glycol graft copolymer and polyvinyl alcohol in combination. Further, a reduction in drug dissolution that occurred when polyvinyl alcohol is used as the coating material was overcame by finding an optimum ratio to the polyvinyl alcohol-polyethylene glycol graft copolymer.

### SUMMARY OF THE INVENTION

Therefore, it is an object of the present invention to provide a composite formulation comprising a film coating layer having excellent tension containing rosuvastatin or a pharmaceutically acceptable salt thereof, which shows reduced defective ratio due to breakage and has excellent dissolution and stability.

In accordance with one aspect of the present invention, there is provided a composite formulation comprising:

(i) a core including a first pharmacological component; and

(ii) a film coating layer formed on a surface of the core, which contains rosuvastatin or its pharmaceutically acceptable salt as a second pharmacological component and a polyvinyl alcohol-polyethylene glycol graft copolymer and polyvinyl alcohol as a coating material.

The composite formulation comprising a film coating layer containing rosuvastatin or its pharmaceutically acceptable salt, a polyvinyl alcohol-polyethylene glycol graft copolymer, and polyvinyl alcohol has low breakage and a defective ratio due to excellent tension and fluidity of the film coating layer, and improved compliance of a patient to a composite agent containing rosuvastatin or the pharmaceutically acceptable salt thereof effective in mitigating and treating a hyperlipemia symptom, and thus useful for the treatment of hyperlipemia.

### BRIEF DESCRIPTION OF THE DRAWINGS

The above and other objects and features of the present invention will become apparent from the following description of the invention, when taken in conjunction with the accompanying drawings, which respectively show:

FIG. 1 shows a cross-section diagram of an exemplary composite formulation according to the present invention, in which a film coating layer containing rosuvastatin is formed on a surface of a core (a: tablet; b: capsule) including a first pharmacological component;

FIG. 2 shows a defective ratio of PTP packaged composite formulations of Preparation Examples 1 to 4 upon discharging the composite formulations from the package;

FIG. 3 shows a dissolution rate of a rosuvastatin calcium salt within 30 minutes for the composite formulations of Preparation Examples 1 to 4;

FIG. 4 shows a defective ratio of PTP packaged composite formulations of Preparation Examples 1 to 4 upon discharging the composite formulations from the package that were stored for one week under 60° C. and 0% RH conditions;

FIG. 5 shows a defective ratio of PTP packaged composite formulations of Preparation Examples 5 to 9 upon discharging the composite formulations from the package;

FIG. 6 shows a dissolution rate of a rosuvastatin calcium salt within 30 minutes for the composite formulations of Preparation Examples 6 to 9;

FIG. 7 shows a defective ratio of PTP packaged composite formulations of Preparation Examples 11 to 13 upon discharging the composite formulations from the package;

FIG. 8 shows a dissolution rate of a rosuvastatin calcium salt within 30 minutes for the composite formulations of Preparation Examples 11 to 13; and

FIG. 9 shows a normal formulation and defective formulations after a defect test.

### DETAILED DESCRIPTION OF THE INVENTION

Hereinafter, the present invention will be described in more detail.

In an exemplary embodiment, the present invention provides a composite formulation comprising a core including a first pharmacological component, and one or a plurality of film coating layers applied on a surface of the core, wherein at least one of the film coating layers includes rosuvastatin or its pharmaceutically acceptable salt, a polyvinyl alcohol-polyethylene glycol graft copolymer, and polyvinyl alcohol, and conforms the external shape of the core.

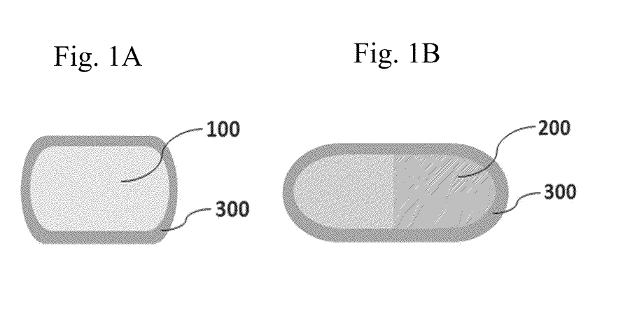

The composite formulation according to the present invention may comprise two parts: the core and the film coating layer (film layer) on the surface of the core. The composite formulation comprising a tablet as the core according to the present invention is illustrated in FIG. 1A, in which reference numeral 100 denotes the tablet core, and reference numeral 300 denotes the film coating layer. The composite formulation comprising a capsule as the core according to the present invention is illustrated in FIG. 1B, in which reference numeral 200 denotes the capsule core, and reference numeral 300 denotes the film coating layer. The drawings show one film coating layer, but it will be understood by a person skilled in the art that a plurality of film coating layers may be devised based on the drawings.

The core may be a formulation including a predetermined first pharmacological component which can be used in combination with a statin-based drug, such as rosuvastatin or its pharmaceutically acceptable salt for improving therapeutic efficiency. Examples of the first pharmacological component may include fenofibric acid or a pharmaceutically acceptable salt thereof, metformin or a pharmaceutically acceptable salt thereof, an omega-3 fatty acid, and ezetimibe, and in one exemplary embodiment of the present invention, fenofibric acid, metformin hydrochloride, or omega-3 fatty acid is used.

The core may be a pharmaceutical formulation typically used in the art, for example, a tablet, a soft capsule, or a hard capsule. The soft capsule or the hard capsule may include two parts of a charge part and a capsule, and each capsule may be a type typically used in the pharmaceutical field. The charge part is a pharmaceutical formulation typically used in the pharmaceutical field, and may be any one of powder, granules, pellets, tablets, and liquid formulations, or a combination of two or more thereof.

The film coating layer may include rosuvastatin or its pharmaceutically acceptable salt as a second pharmacological component, and the polyvinyl alcohol-polyethylene glycol graft copolymer and polyvinyl alcohol as a coating material. The film coating layer may further include any one layer of inner coating, moisture-proof coating, outer coating, enteric coating, sustained-release coating, and photochromic coating layers, or a combination of several layers thereof. Each film coating layer may conform the external shape of the capsule. Examples of the second pharmacological component may include rosuvastatin, rosuvastatin calcium salt, and rosuvastatin magnesium salt, and in one exemplary embodiment of the present invention rosuvastatin calcium salt is used.

The polyvinyl alcohol-polyethylene glycol graft copolymer and polyvinyl alcohol of the film coating layer may be used at a weight ratio of 7:3 to 5:5, and preferably, e.g., 6:4. Further, the coating material including the polyvinyl alcohol-polyethylene glycol graft copolymer and polyvinyl alcohol may be used in an amount of 0.6 to 6 parts by weight, based on 1 part by weight of rosuvastatin or its pharmaceutically acceptable salt, and preferably, e.g., 1 to 4 parts by weight. When the film coating layer includes the polyvinyl alcohol-polyethylene glycol graft copolymer and polyvinyl alcohol in said range, a defective ratio due to breakage of the film coating layer is low, and excellent dissolution and stability can be obtained. The amount of rosuvastatin or its pharmaceutically acceptable salt may be 1 mg to 40 mg in consideration of known daily dosage of rosuvastatin or its pharmaceutically acceptable salt, and preferably, e.g., 1 mg to 20 mg. In the composite formulation of the present invention, the dissolution rate of rosuvastatin or its pharmaceutically acceptable salt is at least 80% within 15 minutes.

The polyvinyl alcohol-polyethylene glycol graft copolymer used in the film coating layer comprises 65% to 85% of a polyvinyl alcohol unit and 15% to 35% of a polyethylene glycol unit, and may comprise about 0.01% to 0.5% of colloidal silica, and has the weight average molecular weight of approximately 35,000 to 55,000 daltons. For example, a representative commercial polyvinyl alcohol-polyethylene glycol graft copolymer, Kollicoat® IR (BASF), comprises about 75% of a polyvinyl alcohol unit and about 25% of a polyethylene glycol unit, and includes about 0.3% of colloidal silica, and has a weight average molecular weight of about 45,000 daltons.

Polyvinyl alcohol used in the film coating layer is a water-soluble polymer, and may have the molecular weight of 20,000 to 200,000 daltons, and viscosity is increased as the molecular weight is increased.

The composite formulation of the present invention may further include a disintegrant, a diluent, a binder, a stabilizer, and a lubricant typically used as a pharmaceutically acceptable additive in addition to the aforementioned components. The pharmaceutically acceptable additive may be used in one or both of the core and the film coating layer.

The disintegrant may be selected from the group consisting of crospovidone, sodium starch glycolate, croscarmellose sodium, low-substituted hydroxypropylcellulose, starch, alginic acid or a sodium salt thereof, and a mixture thereof, but is not limited thereto.

The diluent may be selected from the group consisting of micro-crystalline cellulose, lactose, ludipress, mannitol, monocalcium phosphate, starch, low-substituted hydroxypropylcellulose, and a mixture thereof, but is not limited thereto.

The binder may be selected from the group consisting of hydroxypropylcellulose, hydroxypropylmethylcellulose, polyvinylpyrrolidone, copovidone, macrogol, light anhydrous silicic acid, synthetic aluminum silicate, silicate derivatives such as calcium silicate or magnesium metasilicate aluminate; phosphate salts such as calcium hydrogen phosphate; carbonate salts such as calcium carbonate; and a mixture thereof, but is not limited thereto.

The stabilizer may further increase stability of rosuvastatin or its pharmaceutically acceptable salt. The stabilizer may be an alkaline stabilizer, which may include magnesium carbonate, sodium bicarbonate, sodium carbonate, calcium carbonate, and the like, and, for example, sodium bicarbonate may be used. The alkaline stabilizer may be used in an amount of 0.025 to 5 parts by weight, based on 1 part by weight of rosuvastatin or its pharmaceutically acceptable salt.

The lubricant may be selected from the group consisting of stearic acid, metal stearates such as calcium stearate or magnesium stearate, talc, colloidal silica, sucrose esters of fatty acid, hydrogenated vegetable oils, high melting point waxes, glycerin fatty acid esters, glycerol dibehenate, and a mixture thereof, but is not limited thereto.

The composite formulation according to the present invention may be prepared by a method comprising the step of coating the core including the first pharmacological component with a coating solution including rosuvastatin or its pharmaceutically acceptable salt, the polyvinyl alcohol-polyethylene glycol graft copolymer, and polyvinyl alcohol.

Further, in the preparation method, the coating may be performed by any conventional method known in the art, for example, by a coating method which uses a pan coating device (SFC-30, SEJONG).

The composite formulation of the present invention has low breakage and a defective ratio due to excellent tension and fluidity of the film coating layer containing rosuvastatin or the pharmaceutically acceptable salt thereof. Accordingly, the composite formulation of the present invention has improved compliance of a patient to a composite agent containing rosuvastatin or its pharmaceutically acceptable salt to relieve and treat hyperlipemia symptoms, which is useful in the treatment of hyperlipemia.

Hereinafter, the present invention is described more specifically by the following Examples, but these are provided only for illustration purposes, and the present invention is not limited thereto.

### Preparation Examples 1 to 13

### Preparation of the Composite Formulation Comprising Fenocid Capsule Core Coated with the Film Coating Layer Containing Rosuvastatin Calcium Salt

The coating material having various combined components and amounts as described in the following Table 1, and 1.0 mg of sodium bicarbonate, 13.3 mg of talc, and 10 mg of rosuvastatin calcium salt as an additive were admixed in 1,000 ml of a mixed solution of ethanol and water (ethanol:water=1:1 (v/v)) to prepare a coating solution. The coating solution thus obtained was then coated on to a Fenocid capsule (Hanmi Pharm. Co., Ltd.) including the fenofibric acid as the first pharmacological component, using a pan coating device (SFC-30, SEJONG). The prepared coating capsule was dried at 35° C. for 2 hours to prepare the composite formulation including the Fenocid capsule core coated with the film coating layer containing the rosuvastatin calcium salt.

TABLE 1

| | Rosuvastatin | | | | | | |
|---|---|---|---|---|---|---|---|
| | calcium salt | Coating material (mg) | | | | Coating | Kollicoat ® |
| | (main component) | Kollicoat ® | Polyvinyl | | | material/main | IR:polyvinyl |
| | (mg) | IR | alcohol | Hypromellose | Povidone | component ratio | alcohol ratio |
| Preparation | 10.0 | 25.0 | | | | 2.5 | — |
| Example 1 | | | | | | | |
| Preparation | 10.0 | | 25.0 | | | 2.5 | — |
| Example 2 | | | | | | | |
| Preparation | 10.0 | | | 25.0 | | 2.5 | — |
| Example 3 | | | | | | | |
| Preparation | 10.0 | | | | 25.0 | 2.5 | — |
| Example 4 | | | | | | | |
| Preparation | 10.0 | 20.0 | 5.0 | | | 2.5 | 8:2 |
| Example 5 | | | | | | | |
| Preparation | 10.0 | 17.5 | 7.5 | | | 2.5 | 7:3 |
| Example 6 | | | | | | | |
| Preparation | 10.0 | 15.0 | 10.0 | | | 2.5 | 6:4 |
| Example 7 | | | | | | | |
| Preparation | 10.0 | 12.5 | 12.5 | | | 2.5 | 5:5 |
| Example 8 | | | | | | | |
| Preparation | 10.0 | 10.0 | 15.0 | | | 2.5 | 4:6 |
| Example 9 | | | | | | | |
| Preparation | 10.0 | 3.0 | 2.0 | | | 0.5 | 6:4 |
| Example 10 | | | | | | | |
| Preparation | 10.0 | 6.0 | 4.0 | | | 1.0 | 6:4 |
| Example 11 | | | | | | | |
| Preparation | 10.0 | 24.0 | 16.0 | | | 4.0 | 6:4 |
| Example 12 | | | | | | | |
| Preparation | 10.0 | 36.0 | 24.0 | | | 6.0 | 6:4 |
| Example 13 | | | | | | | |

Kollicoat ® IR: BASF
Polyvinyl alcohol: KURARAY CO., LTD.
Hypromellose 2910 P603: SHIN ETSU CHEMICAL CO., LTD.
Povidone K-30: BASF

### Preparation Example 14

### Preparation of the Composite Formulation Comprising Megaformin Tablet Core Coated with the Film Coating Layer Containing Rosuvastatin Calcium Salt

As described in the following Table 2, 24.0 mg of Kollicoat® IR (BASF) and 16 mg of polyvinyl alcohol (KURARAY CO., LTD.) as the coating materials, and 1.0 mg of sodium bicarbonate, 13.3 mg of talc, and 10 mg of rosuvastatin calcium salt as an additive were admixed in 1,000 ml of a mixed solution of ethanol and water (ethanol:water=1:1 (v/v)) to prepare a coating solution. The coating solution thus obtained was then coated on to a Megaformin tablet (Hanmi Pharm. Co., Ltd.) including metformin hydrochloride as the first pharmacological component, using the pan coating device (SFC-30, SEJONG). The prepared coating tablet was dried at 35° C. for 2 hours to prepare a composite formulation comprising the Megaformin tablet core coated with the film coating layer containing rosuvastatin calcium salt.

TABLE 2

| | Rosuvastatin calcium | Coating material (mg) | | Coating | |
|---|---|---|---|---|---|
| | salt (main component) | Kollicoat ® | Polyvinyl | material/main | Kollicoat ® IR:polyvinyl |
| | (mg) | IR | alcohol | component ratio | alcohol ratio |
| Preparation | 10.0 | 24.0 | 16.0 | 4.0 | 6:4 |
| Example 14 | | | | | |

### Preparation Example 15

### Preparation of the Composite Formulation Comprising Premium Omega-3 Soft Capsule Coated with the Film Coating Layer Containing the Rosuvastatin Calcium Salt

As described in the following Table 3, 24.0 mg of Kollicoat® IR (BASF) and 16.0 mg of polyvinyl alcohol (KURARAY CO., LTD.) as the coating materials, and 1.0 mg of sodium bicarbonate, 13.3 mg of talc, and 10 mg of rosuvastatin calcium salt as an additive were admixed in 1,000 ml of a mixed solution of ethanol and water (ethanol:water=1:1 (v/v)) to prepare a coating solution. The coating solution thus prepared was then coated on to a Premium omega-3 soft capsule (Hanmi medicare, Inc.) including omega-3 fatty acids (EPA and DHA) as the first pharmacologic) component, using the pan coating device (SFC-30, SEJONG). The prepared coating capsule was dried at 35° C. for 2 hours to prepare a composite formulation comprising the Premium omega-3 soft capsule core coated with the film coating layer containing the rosuvastatin calcium salt.

TABLE 3

| | Rosuvastatin calcium | Coating material (mg) | | Coating | |
|---|---|---|---|---|---|
| | salt (main component) | Kollicoat ® | Polyvinyl | material/main | Kollicoat ® IR:polyvinyl |
| | (mg) | IR | alcohol | component ratio | alcohol ratio |
| Preparation | 10.0 | 24.0 | 16.0 | 4.0 | 6:4 |
| Example 15 | | | | | |

### Test Example 1

### Defect Test for Coating Material

The following defect test was performed to evaluate film tension and the breakage ratio of the composite formulations of Preparation Examples 1 to 4.

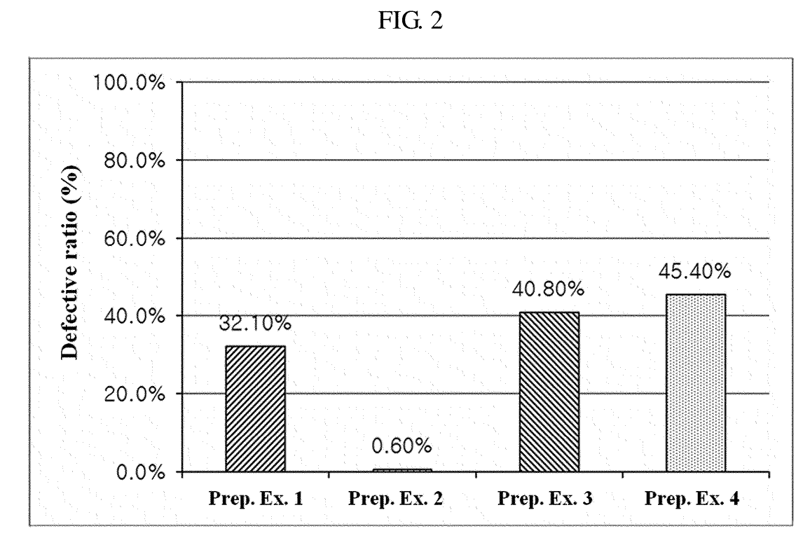

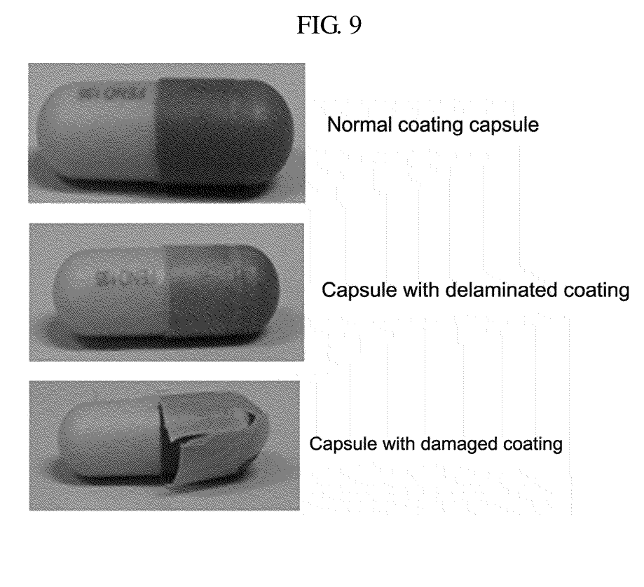

First, an aluminum mold was sealed by an aluminum film using a PTP package machine (Lab-Blister machine, OMAR FANTASY PLUS) to package each composite formulation by PTP. Ten ordinary subjects were randomly selected, and subjected to the test where 100 PTP packages were broken to discharge the packaged drug for each packaged formulation. In this test, as can be seen in FIG. 9, the composite formulation in which the film coating layer was delaminated from the core or damaged was deemed as a defect, and the composite formulation whose coating was well-maintained without any change as compared to its original state before packaging was deemed normal, and the ratio of the number of defects out of a total of 1,000 test groups was checked. The results are shown in the following Table 4 and FIG. 2.

TABLE 4

| | Defective ratio (%) | Coating material |
|---|---|---|
| Preparation Example 1 | 32.1% | Kollicoat ® IR |
| Preparation Example 2 | 0.6% | Polyvinyl alcohol |
| Preparation Example 3 | 40.8% | Hypromellose |
| Preparation Example 4 | 45.4% | Povidone |

From the results of Table 4 and FIG. 2, it could be confirmed that in all of the composite formulations of Preparation Examples 1, 3, and 4 using Kollicoat® IR, hypromellose, and povidone, respectively, as the single coating material, the coating film showed high defective ratios of 30 to 40%. On the contrary, the composite formulation prepared in Preparation Example 2 using polyvinyl alcohol as the single coating material, the defective ratio was 0.6%, which showed an excellent quality.

### Test Example 2

### Dissolution Test for Coating Material

The following dissolution test was performed to evaluate the dissolution rate of rosuvastatin calcium salt of the composite formulations of Preparation Examples 1 to 4.

The dissolution test was performed according to the ‘Dissolution Test’ of the ‘General Test’ of the Korean Pharmacopoeia 10^{th} Edition, and Method 2, i.e., the Paddle method, was used. The Second fluid, i.e., 900 mL of phosphate buffer solution having the pH of 6.8, described in the ‘Disintegration Test’ of the ‘General Test’ of the Korean Pharmacopoeia 10^{th} Edition was used as the test fluid, and the dissolution test was performed at a rate of 50 rpm according to the operation method of the general-release preparation. The test fluid was collected 0 minute, 5 minutes, 10 minutes, 15 minutes, and 30 minutes after the test started, and analyzed according to the ‘Liquid Chromatography’ of the ‘General Test’ of the Korean Pharmacopoeia 10^{th} Edition to obtain the dissolution rate at the corresponding point by comparing it with the standard fluid which was prepared in advance.

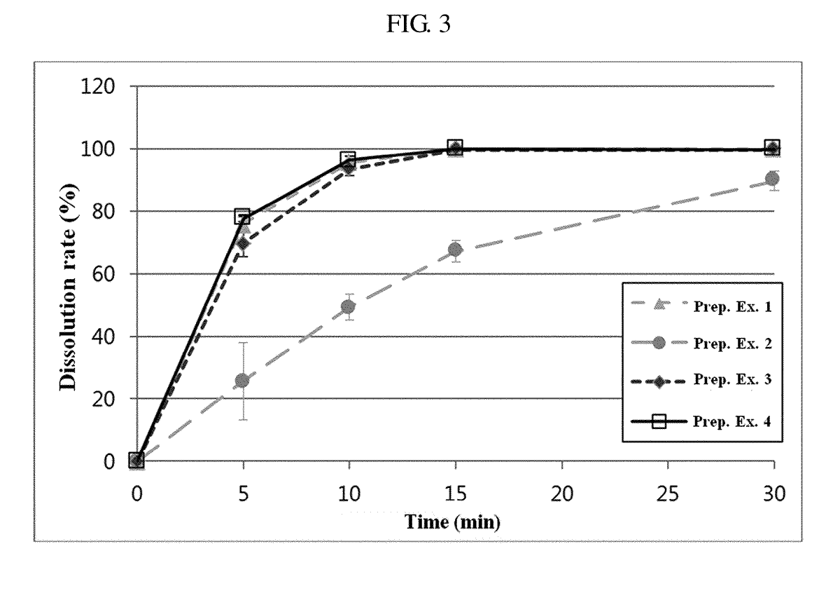

The results are shown in the following Table 5 and FIG. 3.

TABLE 5

| | Preparation Example 1 | | Preparation Example 2 | | Preparation Example 3 | | Preparation Example 4 | |
|---|---|---|---|---|---|---|---|---|
| Time | | Standard | | Standard | | Standard | | Standard |
| (min) | Average | deviation | Average | deviation | Average | deviation | Average | deviation |
| 0 | 0 | 0 | 0 | 0 | 0 | 0 | 0 | 0 |
| 5 | 75.9 | 2.3 | 25.6 | 12.4 | 69.7 | 4.2 | 77.6 | 1.1 |
| 10 | 95.6 | 0.4 | 49.3 | 4.1 | 93.5 | 2.2 | 96.3 | 1.3 |
| 15 | 99.8 | 0.2 | 67.2 | 3.4 | 99.6 | 0.1 | 99.9 | 0.2 |
| 30 | 99.9 | 0.2 | 89.7 | 3.2 | 99.6 | 0.1 | 99.8 | 0.1 |

From the results of Table 5 and FIG. 3, it can be confirmed that the composite formulations of Preparation Examples 1, 3, and 4 using Kollicoat® IR, hypromellose, and povidone, respectively, have the relatively high dissolution rates as compared to the composite formulation of Preparation Example 2 using polyvinyl alcohol.

Rosuvastatin calcium salt typically available on the market is an immediate-release or general-release tablet, and has a high dissolution rate of 90% or more within 15 minutes, and thus the rosuvastatin calcium salt is rapidly absorbed at an early stage of administration. Thus, it is important for the immediate-release formulation of rosuvastatin calcium salt to have a high dissolution rate within 15 minutes.

### Test Example 3

### Defect Test for Coating Material Over Time

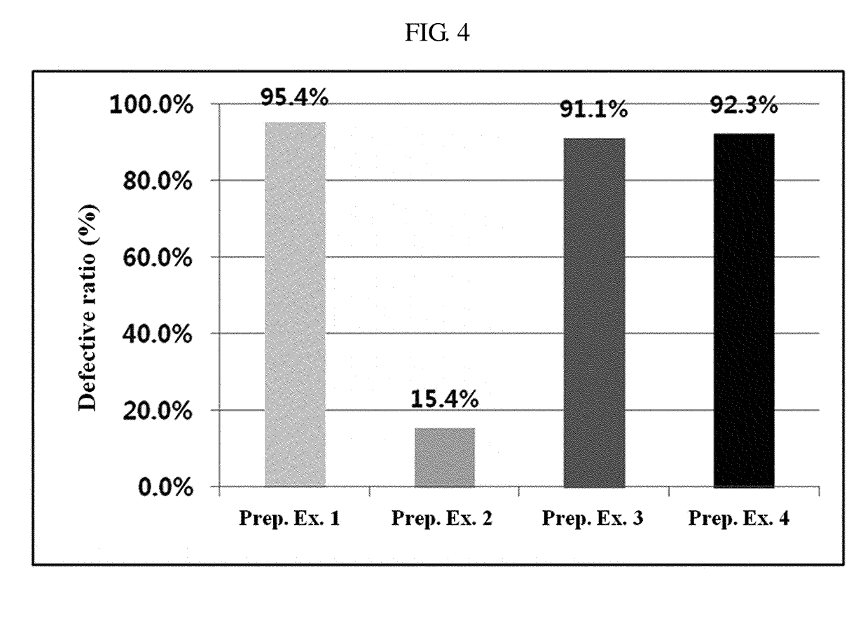

In order to evaluate stability according to the storage time of the composite formulations of Preparation Examples 1 to 4, the each sample was stored in a constant-temperature chamber at 60° C. and RH of 0% for one week, and then subjected to a defect test according to the same method as Test Example 1. The test results are shown in the following Table 6 and FIG. 4.

TABLE 6

| | Defective ratio (%) | Coating material |
|---|---|---|
| Preparation Example 1 | 95.4% | Kollicoat ® IR |
| Preparation Example 2 | 15.4% | Polyvinyl alcohol |
| Preparation Example 3 | 91.1% | Hypromellose |
| Preparation Example 4 | 92.3% | Povidone |

From the results of Table 6 and FIG. 4, it can be seen that in all of the formulations of Preparation Examples 1, 3, and 4 using Kollicoat® IR, hypromellose, and povidone, respectively, as the single coating material, moisture was completely removed under the dry condition of the high temperature for one week, and thus the film coating layer was damaged at a high defective ratio of 90% or more. On the contrary, the formulation obtained in Preparation Example 2 using polyvinyl alcohol as the single coating material had a defective ratio of 15.4% which was increased as compared to the result of Test Example 1, but it demonstrated relatively satisfactory ability in film property maintenance as compared to the other Preparation Examples.

In consideration of the results of Test Examples 1 to 3, it can be seen that there is a need for a formulation with high stability and low defective ratio which can overcome the low dissolution rate of the formulation of Preparation Example 2.

### Test Example 4

### Defect Test of Formulation Comprising Polyvinyl Alcohol-Polyethylene Glycol Graft Copolymer and Polyvinyl Alcohol

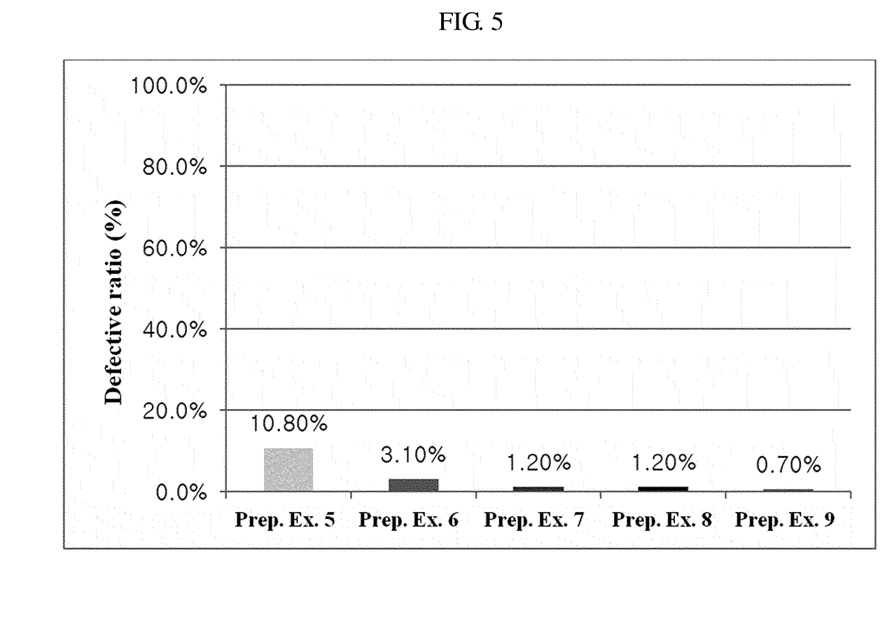

The formulations of Preparation Examples 5 to 9 were subjected to a defect test according to the same conditions and method as Test Example 1, and the results are shown in the following Table 7 and FIG. 5.

TABLE 7

| | | Kollicoat ® IR:polyvinyl |
|---|---|---|
| | Defective ratio (%) | alcohol ratio |
| Preparation Example 5 | 10.8% | 8:2 |
| Preparation Example 6 | 3.1% | 7:3 |
| Preparation Example 7 | 1.2% | 6:4 |
| Preparation Example 8 | 1.2% | 5:5 |
| Preparation Example 9 | 0.7% | 4:6 |

From the results of Table 4 and FIG. 2 and Table 7 and FIG. 5, it can be seen that in the case of Preparation Examples 5 to 9 using Kollicoat® IR and polyvinyl alcohol in combination, the defective ratio was significantly reduced as compared to Preparation Example 1 using only Kollicoat® IR and Preparation Example 2 using only polyvinyl alcohol.

However, when the defective ratio largely exceeds about 3%, a manufacturer may suffer from a loss during manufacturing of drugs and a consumer may have complaints during distribution of drugs. Accordingly, Preparation Examples 6 to 9 using Kollicoat® IR and polyvinyl alcohol at a ratio of 7:3 to 4:6 could be considered to be preferable as compared to Preparation Example 5 using Kollicoat® IR and polyvinyl alcohol at a ratio of 8:2.

### Test Example 5

### Dissolution Test of Formulation Comprising Polyvinyl Alcohol-Polyethylene Glycol Graft Copolymer and Polyvinyl Alcohol

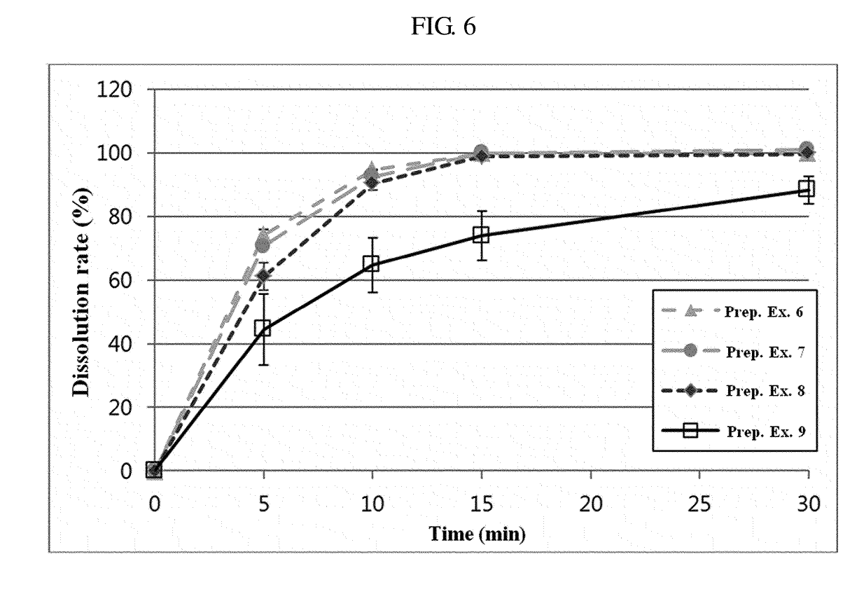

The formulations of Preparation Examples 6 to 9 were subjected to a dissolution test according to the same conditions and method as Test Example 2, and the results are shown in the following Table 8 and FIG. 6.

TABLE 8

| | Preparation Example 6 | | Preparation Example 7 | | Preparation Example 8 | | Preparation Example 9 | |
|---|---|---|---|---|---|---|---|---|
| Time | | Standard | | Standard | | Standard | | Standard |
| (min) | Average | deviation | Average | deviation | Average | deviation | Average | deviation |
| 0 | 0 | 0 | 0 | 0 | 0 | 0 | 0 | 0 |
| 5 | 74.2 | 1.7 | 70.7 | 1.0 | 61.1 | 4.2 | 44.5 | 11.2 |
| 10 | 94.7 | 0.1 | 92.5 | 0.5 | 90.4 | 2.2 | 64.7 | 8.6 |
| 15 | 99.8 | 0.2 | 99.9 | 0.2 | 98.9 | 0.1 | 73.9 | 7.7 |
| 30 | 99.8 | 0.1 | 101.1 | 0.2 | 99.6 | 0.1 | 88.3 | 4.2 |

From the results of Table 8 and FIG. 6, it can be seen that the formulation of Preparation Example 9 using Kollicoat® IR and polyvinyl alcohol at a weight ratio of 4:6 has a significantly reduced dissolution rate as compared to Preparation Example 8 using Kollicoat® IR and polyvinyl alcohol at a weight ratio of 5:5. Accordingly, the formulations of Preparation Examples 6 to 8 could be considered to be preferable since the sufficient dissolution was ensured as the immediate-release or general-release formulation and the defective ratio was low, as confirmed in Test Example 4.

### Test Example 6

### Defect Test for Formulations in Various Ratios of Coating Material Comprising Polyvinyl Alcohol-Polyethylene Glycol Graft Copolymer and Polyvinyl Alcohol to Rosuvastatin Calcium Salt

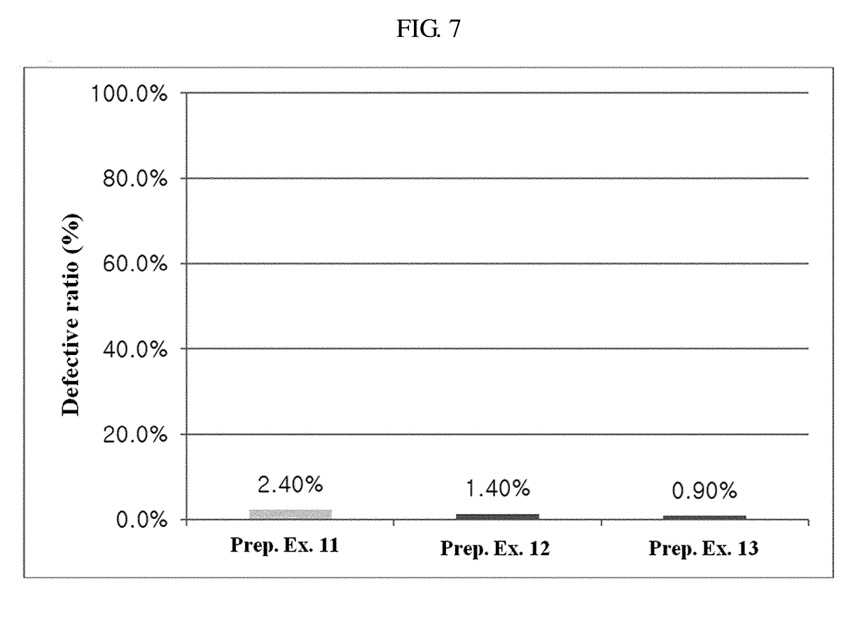

For Preparation Examples 10 to 13 in which the ratio of Kollicoat® IR and polyvinyl alcohol was fixed to 6:4, the same ratio as in Preparation Example 7 while the ratio of the coating material to the second pharmacological component, i.e., rosuvastatin calcium salt, was changed, the defect test was performed according to the same conditions and method as Test Example 1, and the results are shown in the following Table 9 and FIG. 7.

TABLE 9

| | | Coating | |
|---|---|---|---|
| | | material/second | |
| | Defective ratio | pharmacological | Formation |
| | (%) | component ratio | of film |
| Preparation Example | — | 0.5 | X |
| 10 | | | |
| Preparation Example | 2.4% | 1.0 | ◯ |
| 11 | | | |
| Preparation Example | 1.4% | 4.0 | ◯ |
| 12 | | | |
| Preparation Example | 0.9% | 6.0 | ◯ |
| 13 | | | |

From the results of Table 9 and FIG. 7, it can be seen that the defective ratio was low for each ratio, but the formulation of Preparation Example 10 having a coating material/second pharmacological component ratio of 0.5 exhibited poor film formation due to shortage of the coating material and thus coating was not readily performed. Accordingly, the film formation could be considered to be preferable when the coating material/second pharmacological component ratio of 1.0 or more.

### Test Example 7

### Dissolution Test for Formulations in Various Ratios of Coating Material Comprising Polyvinyl Alcohol-Polyethylene Glycol Graft Copolymer and Polyvinyl Alcohol to Rosuvastatin Calcium Salt

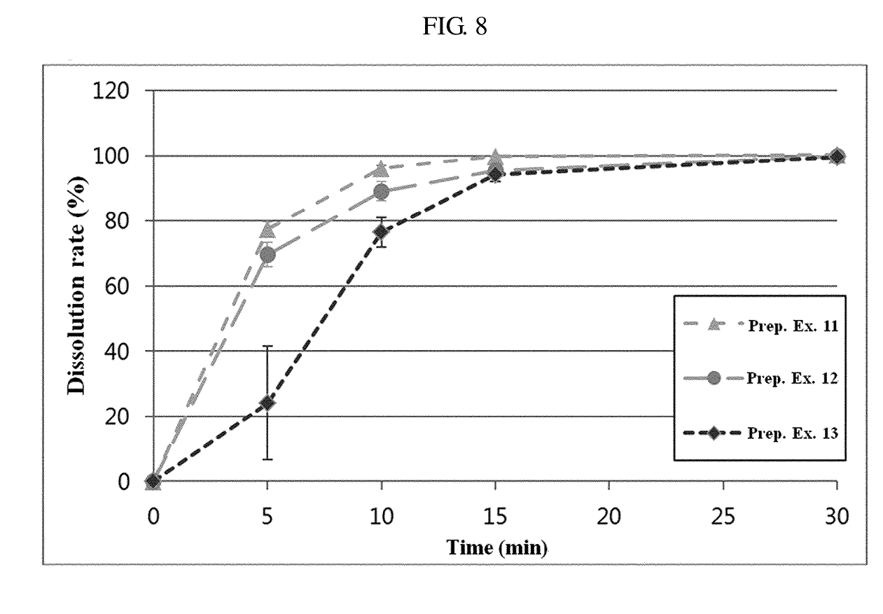

For Preparation Examples 11 to 13, a dissolution test was performed according to the same conditions and method as Test Example 2, and the results are shown in the following Table 10 and FIG. 8.

TABLE 10

| | Preparation | | Preparation | | Preparation | |
|---|---|---|---|---|---|---|
| | Example 11 | | Example 12 | | Example 13 | |
| Time | Aver- | Standard | Aver- | Standard | Aver- | Standard |
| (min) | age | deviation | age | deviation | age | deviation |
| 0 | 0 | 0 | 0 | 0 | 0 | 0 |
| 5 | 77.4 | 2.5 | 69.7 | 3.8 | 24.1 | 17.4 |
| 10 | 96.2 | 0.7 | 89.1 | 3 | 76.5 | 4.6 |
| 15 | 99.8 | 0.2 | 95.4 | 1.1 | 94.2 | 2.2 |
| 30 | 100.2 | 0.1 | 99.8 | 0.2 | 99.6 | 0.1 |

From the results of Table 10 and FIG. 8, it could be seen that in the formulation of Preparation Example 13 having the ratio of the coating material to the main component of 6.0, a significant reduction in initial dissolution occurred and a deviation in dissolution was large.

Accordingly, the formulations of Preparation Examples 11 and 12 having the sufficient dissolution rate for immediate-release or general-release formulation could be considered to be preferable.

### Test Example 8

### Defect Test of Formulations Comprising Polyvinyl Alcohol-Polyethylene Glycol Graft Copolymer and Polyvinyl Alcohol in Various Core Types

The formulations of Preparation Examples 12, 14, and 15 were subjected to a defect test according to the same conditions and method as Test Example 1, and the results are shown in the following Table 11

TABLE 11

| | Defective | | Coating |
|---|---|---|---|
| | ratio | Kollicoat ® IR:polyvinyl | material/main |
| | (%) | alcohol ratio | component ratio |
| Preparation | 1.4% | 6:4 | 4.0 |
| Example 12 | | | |
| Preparation | 0.2% | 6:4 | 4.0 |
| Example 14 | | | |
| Preparation | 1.3% | 6:4 | 4.0 |
| Example 15 | | | |

From the result of Table 11, it could be seen that both the tablet of Preparation Example 14 and the soft capsule of Preparation Example 15 had excellent properties and low defective ratios when the same coating as Preparation Example 12 having the excellent film property and dissolution rate was applied to the cores, regardless of their core types.

Accordingly, it can be concluded that the film coating layer of the present invention could be applied not only to hard capsule cores, but also to cores having various types as well.

What is claimed is:

1. A composite formulation comprising:
(i) a core including a first pharmacological component; and
(ii) a film coating layer formed on a surface of the core, which contains rosuvastatin or a pharmaceutically acceptable salt thereof as a second pharmacological component and a polyvinyl alcohol-polyethylene glycol graft copolymer and polyvinyl alcohol as a coating material.

2. The composite formulation of claim 1, wherein the first pharmacological component is fenofibric acid or a pharmaceutically acceptable salt thereof, metformin or a pharmaceutically acceptable salt thereof, an omega-3 fatty acid, or ezetimibe.

3. The composite formulation of claim 1, wherein the core is a tablet, a soft capsule, or a hard capsule.

4. The composite formulation of claim 1, wherein the second pharmacological component is rosuvastatin calcium salt.

5. The composite formulation of claim 1, wherein the polyvinyl alcohol-polyethylene glycol graft copolymer and polyvinyl alcohol of the film coating layer is used at a weight ratio of 7:3 to 5:5.

6. The composite formulation of claim 1, wherein the formulation comprises the coating material in an amount ranging from 1 to 4 parts by weight, based on 1 part by weight of rosuvastatin or its pharmaceutically acceptable salt.

7. The composite formulation of claim 1, wherein the formulation comprises rosuvastatin or its pharmaceutically acceptable salt in an amount ranging from 1 mg to 40 mg.

8. The composite formulation of claim 1, wherein a dissolution rate of rosuvastatin or the pharmaceutically acceptable salt thereof is at least 80% for 15 minutes.

9. The composite formulation of claim 1, wherein one or more of the core and the film coating layer comprise a pharmaceutically acceptable additive.

10. The composite formulation of claim 9, wherein the additive is at least one selected from the group consisting of a disintegrating agent, a diluent, a binder, a stabilizer, and a lubricant.

11. The composite formulation of claim 1, wherein the film coating layer further comprises an alkaline stabilizer.

12. The composite formulation of claim 11, wherein the alkaline stabilizer is magnesium carbonate, sodium bicarbonate, sodium carbonate, or calcium carbonate.

13. The composite formulation of claim 11, wherein the alkaline stabilizer is used in an amount of 0.025 to 5 parts by weight, based on 1 part by weight of rosuvastatin or its pharmaceutically acceptable salt.
